(19) United States
(12) Patent Application Publication
(10) Pub. No.: US 2025/0266147 A1
(43) Pub. Date: Aug. 21, 2025

(54) SYSTEM AND METHOD FOR PROMOTING, TRACKING, AND ASSESSING MENTAL WELLNESS

(71) Applicants: Alexandria Brown SKALTSOUNIS, West Hollywood, CA (US); Clenét VERDI-ROSE, Los Angeles, CA (US)

(72) Inventors: Alexandria Brown SKALTSOUNIS, West Hollywood, CA (US); Clenét VERDI-ROSE, Los Angeles, CA (US)

(21) Appl. No.: 19/198,359

(22) Filed: May 5, 2025

Related U.S. Application Data
(63) Continuation-in-part of application No. 17/978,571, filed on Nov. 1, 2022.
Application No. 17/978,571 is a continuation-in-part of application No. 17/183,673, filed on Feb. 24, 2021, now Pat. No. 11,521,715.

(51) Int. Cl. G16H 20/70; A61B 5/00; A61B 5/16; G16H 10/60; G16H 50/30

(52) U.S. Cl. CPC G16H 20/70; A61B 5/0013; A61B 5/0077; A61B 5/165; A61B 5/4803; A61B 5/7246; A61B 5/7435; A61B 5/7475; G16H 10/60; G16H 50/30

(57) ABSTRACT

A method and system for data transmission include inputting an entry from a user through an interface on a user device; transmitting the entry to a remote data storage; storing the entry with user historical data; analyzing, with a processor, the entry; performing correlations on the entry using at least the user historical data; determining at least one of entry classifications, baselines, or a new input prompt as a result of correlated entry data; transmitting the at least one of entry classifications, baselines, or new input prompt to the user device; and displaying the at least one of entry classifications, baselines, or new input prompt on the user device. Further, the method and system include performing correlations on the entry using cohort data, transmitting a notification to a supervisory user, and determining and generating threshold values associated with at least one of historical user data and cohort data.

### CROSS-REFERENCE TO RELATED APPLICATIONS

This application is a continuation-in-part of U.S. patent application Ser. No. 17/978,571, filed on Nov. 1, 2022 which is a continuation-in-part of U.S. patent application Ser. No. 17/183,673, filed on Feb. 24, 2021.

### BACKGROUND

While the focus on mental health has increased in recent decades, mental health resources utilizing technological advancements to provide insight into human development, emotion, and state of mind are frequently overlooked. Proactive and impartial mental health solutions are still lacking and it can be difficult to express and quantify how one may truly feel. For example, a child may be shy or not willing to express their actual feelings depending on the contextual circumstances. Furthermore, comprehensive solutions for tracking a subject user's mental health, moods, and feelings over extended terms of time, and for providing such information to supervisory users (e.g., clinicians) are also lacking. A solution that can provide a calm and nurturing place to practice positive mental health techniques and advance the collection of mental health data and analytics without bias and prejudice of analysis is therefore needed. A solution that can provide parents, caretakers, and professionals with detailed insight and analysis of the mental health of a subject user while providing contextual content management and streamlining traditional processes is therefore needed.

### SUMMARY

An embodiment includes method for data transmission includes inputting an entry from a user through an interface on a user device; transmitting the entry to a remote data storage; storing the entry with user historical data; analyzing, with a processor, the entry; performing correlations on the entry using at least the user historical data; determining at least one of entry classifications, baselines, or a new input prompt as a result of correlated entry data; transmitting the at least one of entry classifications, baselines, or new input prompt to the user device; and displaying the at least one of entry classifications, baselines, or new input prompt on the user device.

Another embodiment includes a communication system. The communication system includes a user input device configured for wireless communication to at least a remotely located data storage; and a supervisory communication device configured for wireless communication to at least the remotely located data storage. The communication system further includes a processor configured to: receive input data from the user device; analyze input data; compare the input data with one or more of historical user data and cohort data; determine when the input data are at least one of entry classifications, baselines, or exceeds a threshold; generate new entry classifications, baselines, or an input prompt when the data exceeds the threshold; transmit at least one of entry classifications, baselines, or the input prompt to the user device; classify the at least one of entry classifications, baselines, or input data; and initiate a synchronous communication session between the user device and a supervisory user or generate a dynamically populated response that is transmitted to the user device from a supervisory user when the data exceeds the threshold.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 shows an exemplary system for promoting, tracking, and assessing mental health and wellness.

FIGS. 2a-2d show exemplary interfaces of the computer program product for promoting, tracking, and assessing mental health and wellness.

FIG. 3 shows an exemplary method for receiving mental wellness information.

FIG. 4 shows an exemplary method for analyzing mental wellness information.

FIG. 5 shows an exemplary user timeline with conflicts tagged.

FIG. 6 shows an exemplary contextual mesh timeline.

FIG. 7. shows an exemplary behavioral baseline structured prompt map.

FIG. 8 shows an exemplary user-behavioral analysis system.

### DETAILED DESCRIPTION

Aspects of the invention are disclosed in the following description and related drawings directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many of the embodiments described herein may be described in terms of sequences of actions to be performed by, for example, elements of a computing device. It should be recognized by those skilled in the art that the various sequence of actions described herein can be performed by specific circuits (e.g., application specific integrated circuits (ASICs)) and/or by program instructions executed by at least one processor. Additionally, the sequence of actions described herein can be embodied entirely within any form of computer-readable storage medium such that execution of the sequence of actions enables the processor to perform the functionality described herein. Thus, the various aspects of the present invention may be embodied in a number of different forms, all of which have been contemplated to be within the scope of the claimed subject matter. In addition, for each of the embodiments described herein, the corresponding form of any such embodiments may be described herein as, for example, “a computer configured to” perform the described action.

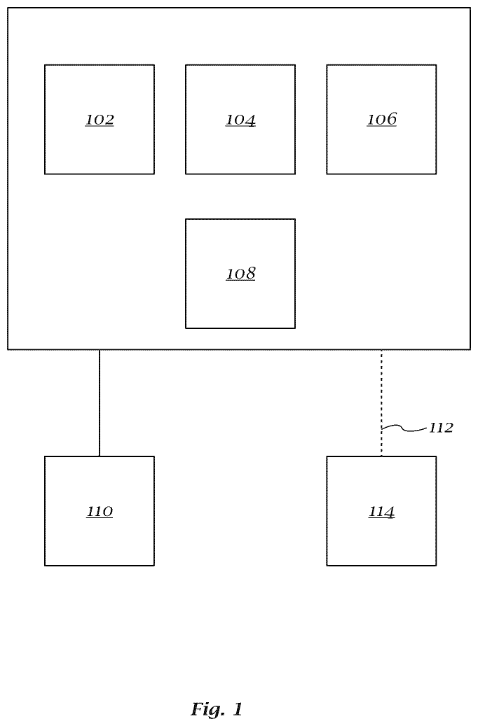

According to at least one exemplary embodiment, a diagnostic system and method for promoting, tracking, and assessing mental health and wellness 100 is disclosed. As shown in FIG. 1, system 100 may include a plurality of modules that may be interacted with by a subject user or a supervisory user such as a parent, caretaker, or medical professional. For example, system 100 may include an entry module 102, an exercise module 104, and an analytics module 106. System 100 may further include one or more data storages 108, which may be any data storage and management implementation known in the art. In some exemplary embodiments, system 100 may be provided on a user-side computing device 110, such as, for example, as an application for a computer or a mobile device. In such embodiment, the various modules of system 100 and the data storage 108 may be present on the user-side computing device 110 and executed on device 110. In other exemplary embodiments, all or portions of system 100 may be provided on a cloud or communication network 112, which may be a wired or wireless network implemented via any communication technique known in the art, and provide for the transmission of data (such as input data) from device 110 to any other device or storage, as desired. For example, data store 108 and analytics module 106 may be provided on a server side 114, while entry module 102 and exercise module 104 may be provided on the user-side computing device 110. In yet other exemplary embodiments, all components may be provided on the server side 114, and system 100 may be accessible via interfaces provided on user-side computing devices 110, such as, for example, a web-based application or a standalone application. Further, any inputs or interaction between a user and the system 100 can be automatically transmitted and remotely stored in real time during an interaction, or transmitted/uploaded to network 112 immediately following the interaction or use of the system 100. Further, in some embodiments, the system 100 may automatically classify some interactions as being above or below a threshold (such as a “negativity threshold”) and may automatically act to store those interactions remotely immediately upon the interaction or use taking place. Further, the threshold, such as the negativity threshold, may be automatically generated or determined, for example using artificial intelligence or machine learning that is trained based on historical user information, using historical or real time cohort information, or a combination of user historical information and cohort information.

In some exemplary embodiments, entry module 102 and exercise module 104 may be oriented towards interaction with young children, for example, children who have not yet learned to write, or children in the 2-8-year-old range. For example, entry module 102 may include interfaces to allow a child to draw or speak to record their moods and feelings. In further exemplary embodiments, entry module 102 may also include interfaces for recording clinical sessions, for example between the subject user and a mental health counselor or other clinical professional. Exercise module 104 may include interactive, guided exercises to teach mental health and wellness principles. The various exercises of the exercise may include animated characters that speak and move to provide guidance for the child as to how to perform the exercises. For example, the exercises may include breathing exercises, mood exercises, guided relaxation and meditation, body exercises, empathy lessons showing how feelings manifest in the body, emotion identification lessons for autistic children, exercises for cultivating imagination, healthy nutrition and wellness habit lessons, and sound programs for aiding sleep. Further, the animated characters who provide guidance may be automatically generated by a processor and/or using AI, for example in a color tone or palette that promotes positivity or good feelings, for example to counteract mood inputs or cues received by the system. In further embodiments, the character's forms may be adopted by trained AI to provide customized responses, therapeutic recommendations, generate exercises, or other suggestions. In certain embodiments of clinical application, the supervisory user may adopt the form of the character or avatar to dynamically connect with the user in real time (i.e., a virtual therapy portal session and a therapist speaking as a character to calm or connect to a child.) In such embodiments, the character or avatar may mimic motions, actions, or expressions of a supervisory user, for example through motion capture technology. Analytics module 106 may provide interfaces and data analysis for parents, caretakers, health professionals, and the like. Analytics module 106 may utilize data obtained at least from entry module 102 to track a child's moods and mental health over time. In yet further exemplary embodiments, the modules of system 100 may be oriented towards interactions with subject users of different ages or needs. For example, the modules of system 100 may be oriented towards pre-teen users (i.e., ages 9-13), teenage users, adults experiencing PTSD, dementia, or other disabilities or illnesses. System 100 may further be utilized to aid in various settings, for example individual or group counseling sessions for various issues (for example, anger management, substance dependence, mental illness, and so forth). Additionally, system 100 may be further utilized in conjunction with algorithms, for example artificial intelligence algorithms, to provide further insight, track, or corroborate emotional verification or dissonance for a statement, opinion, or testimony.

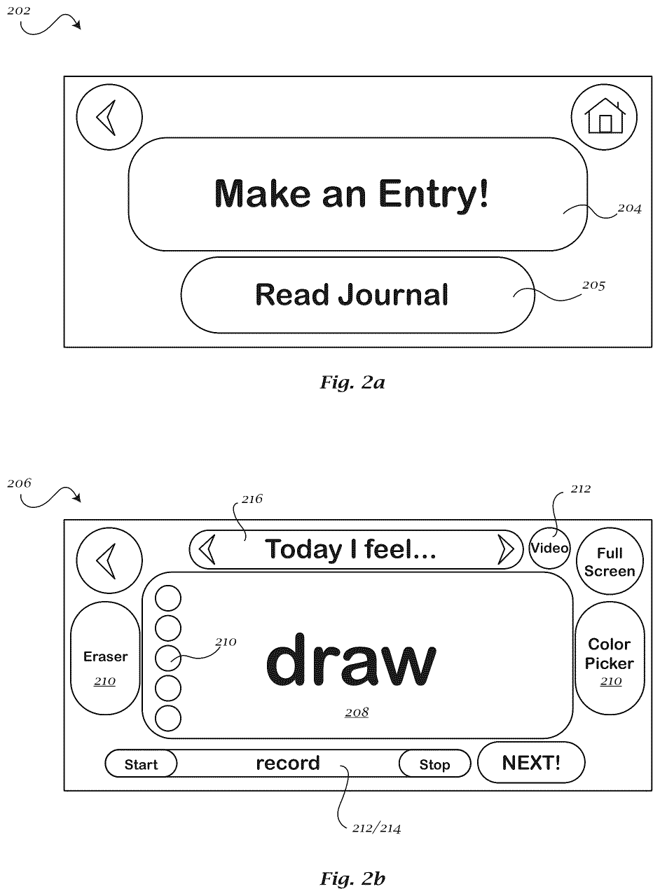

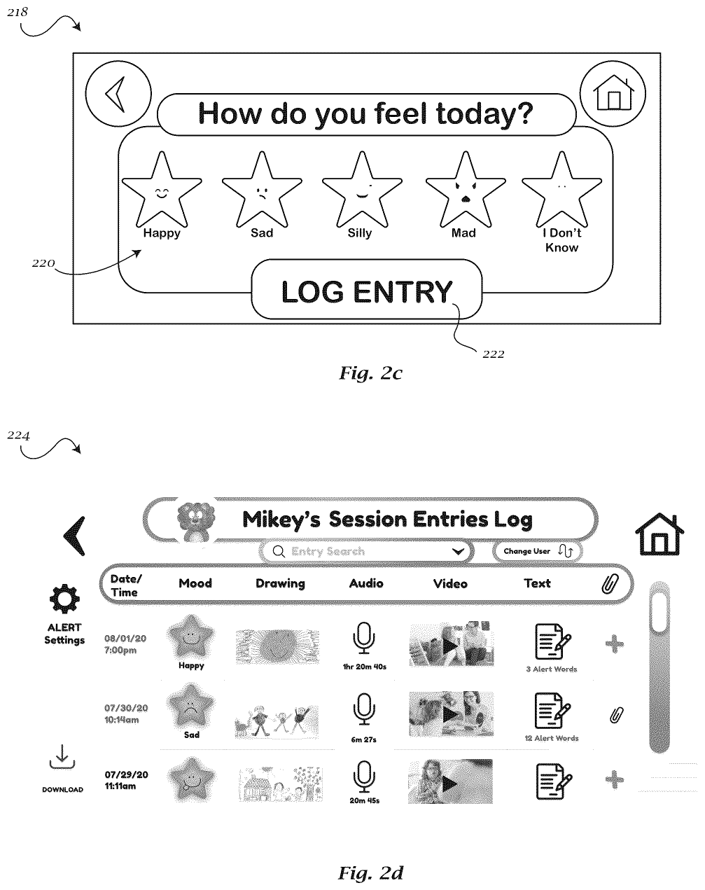

Turning to FIG. 2a, the entry module may include a plurality of interfaces for interaction with subject users. In such embodiments, the entries may include journal entries. The entry module can be adapted to receive drawn, written, spoken, and/or video input from the user. Interfaces of the entry module may be adapted to provide easy navigation and prompting to allow ease of interaction for subject users of system 100. In an exemplary embodiment, which may be adapted towards direct interaction with subject users, a first interface 202 may include options for making a journal entry 204 or reading journal entries 205. If the subject user selects to make a journal entry, an input interface 206 may be provided, as shown in FIG. 2b. The input interface 206 may include input tools such as a canvas 208, drawing and text tools 210, video record 212, and audio record 214. Furthermore, input interface 206 may include a journal prompt 216. Journal prompt 216 may present the subject user with a prompt for the journal entry. A default prompt may be initially presented and the subject user may select from a variety of additional prompts as well. For example, such prompts may include “Today I feel . . . ”, “I am grateful for . . . ”, “I got upset because . . . ”, “I like myself because . . . ”, “My dream is to . . . ”, “I showed kindness when . . . ”, and so forth. Journal entry prompts may be preloaded in the entry module, and custom prompts may also be created by a supervisory user. Further, depending on past data or other known conditions, the supervisory user may preload or initiate a prompt 216 for a user. Additionally, journal prompts 216 may be automatically or dynamically generated based on past or current inputs by a user. For example, the system 100, using trained AI, may learn or understand that a user is experiencing negative emotions and may provide a prompt 216 that focuses on positivity. Furthermore, calendars of prompts may be created by the supervisory user or created by AI and adopted by a supervisory user in a treatment plan. Moreover, trained AI may identify an emerging trendline and guide prompts to provide information to a supervisory user or clinician. Additionally, the system may identify and dynamically initiate structured prompt flows or “worksheet” structures common in clinical application (i.e., Stressful Incident Prompt Structures, CBT Techniques, or worksheets specific to certain mental illnesses such as Anxiety or PTSD) to assess threshold weight and frequency of occurrence. In the context of collecting context or testimony over time, AI prompts may provide structured impartial guidance through accounts of events and experiences or testimony questions. In the context of advanced clinical use, AI may provide treatment specific prompt sequences over time that are specific to a Therapeutic Method (i.e. Psychoanalytic, Behavioral, Humanistic, Transpersonal, or Art Therapy, Exposure Therapy, etc.), a specific therapist's methodology, diagnostic trendline, and the specific needs of the individual user. Furthermore throughout the users inputs to the system, AI may identify an area which may need to be further elaborated upon or needs deeper investigation and may suggest prompts to provide more data such as historical accounts of events. (i.e. discovering the need to look deeper at traumatic instances or formative memories providing robust background historical analysis.) For example, an adult who is scared of clowns thinks of an old memory where they saw a movie playing in the living room late at night about clowns, the need to learn more about this memory may provide important clinical insight. Additionally, the system 100 can dynamically generate and provide prompts 216 in real time as user inputs or cues are entered and processed by the system 100. However, in some circumstances, the subject user may select a desired prompt and then create a journal entry with the available input tools, such as the canvas 208 with drawing and text tools 210, video record 212, and audio record 214. Once the subject user has completed the journal entry using one or more of the input tools, a subsequent interface may be displayed by way of operation of a control such as a next button or the like.

In some exemplary embodiments, the subsequent interface may be a mood interface 218, as shown in FIG. 2c. In the mood interface 218, the subject user may be prompted to choose a mood from a plurality of mood indicators 220, such as “happy”, “sad”, “silly”, “mad”, “I don't know”, and so forth. Mood indicators may be preloaded in the entry module, and custom moods may also be created by a supervisory user. In some exemplary embodiments, the subject user may also be prompted to select a color from a spectrum of colors that the subject user feels matches their mood. After the subject user selects a mood indicator 220 and/or chooses a color, the subject user may log their entry by log entry control 222. The journal entries, including the drawn, text, and/or recorded inputs, along with the mood of the subject user may then be saved to data storage.

In yet further exemplary embodiments, entry module 102 may be adapted towards recording sessions between a subject user and a clinical professional, who may also be a supervisory user. In such embodiments, the entries of entry module 102 may include recordings of entire sessions, or portions of sessions, between the subject user and the clinical professional. The sessions may be logged, including the time, place, and duration of the session. Sessions may take place as remote sessions, with video and/or audio interaction being provided by system 100 on the computing devices or mobile devices of the subject user and the clinical professional. In addition, screen sharing functionality between the subject user and the clinical professional may be provided by system 100, such that both users can view a common interface on which interactions may be performed, including drawing, text input, mood selection, and so forth. Sessions may also take place as in-person sessions, with system 100 providing audio and/or video recording functionality of the session. Furthermore, session recordings (drawn, written, audio or video), and/or transcripts may be submitted from sources external to system 100 and may be classified as subject-user-submitted, supervisory-user-submitted, or other sessions by system 100. Subsequent to the recording of a session, the subject user may then be provided with mood interfaces, as described above.

An entry log interface 224, for example as shown in FIG. 2d, may allow a user of system 100 to review past entries (i.e., journal and/or session entries), including the date and time the entry was logged, the mood, drawing, text, audio recording, and or video recording of the entry. A subject user or supervisory user may select any of the logged inputs for an entry to view the contents thereof. System 100 may also be provided with speech-to-text functionality adapted to transcribe contents of the audio and video recordings. The transcripts of the audio and video recordings may be provided with each entry.

In some exemplary embodiments, entry module 102 may provide additional features. For example, users such as supervisory users may be able to add notes, attachments, flags, and/or forms to any entry for future reference by the supervisory user. Notes may be available to be added to all inputs, i.e., logged drawings, text, videos, audio recordings, and transcriptions, and may be added to any temporal or spatial location in the input. Attachments may further be added to an entry, so as to provide comprehensive context for the entry. An attachment may be a document of any format, for example, text, image, video, PDF, and so forth. For example, if the subject user is a child, attachments may include items relevant to the particular entry of the subject user, such as report cards, social media posts, school projects or assignments, disciplinary items, and so forth. With respect to sessions, such attachments may include any forms from the clinical professional that are relevant to the session, any comments by the clinical professional on the session, and so forth. Such attachments may aid supervisory and subject users in creating a comprehensive log that may be reviewed subsequently or in a professional counseling context. Additionally, supervisory users may flag entries so as to provide further context for the entry. For example, a flag may be added to indicate that the entry was part of a high-stress incident in a subject user's life, a time-out, detention, episode, or so forth. Conversely, a flag may be added to indicate that the entry was part of a low-stress or pleasurable time in the subject user's life, such as a celebration, accomplishment, vacation, and so forth.

In some exemplary embodiments, supervisory users may be provided with interfaces directed towards features useful in a clinical environment. For example, such a clinical interface can facilitate maintaining audio and/or video recordings of sessions, which can then be associated to a user as entries for that user. The clinical entries can then be transcribed and analyzed by system 100 as described herein. The clinical interface can further provide for recording of both in-person and remote sessions. Additional features that may be provided by the clinical interface can include form creation and management, virtual waiting room, virtual chat with interactive features, video chat, fidget toggles, schedule management, diagnostic quizzes, and so forth.

A search feature may allow supervisory users to review the entries and associated notes and attachments and to determine trends. Searches may be performed by date and time, duration of recording, mood, entry content, number of alert words per entry or sequences of alert words per entry, specific alert words or sequences of alert words, or the like. The search feature may be able to search in real time, and may further include searches for trendlines, doctor provided diagnosis, commonality variables, mood over time, and/or other meta data. Alert settings may further be provided. For example, a supervisory user can define alerts based on a keyword, a mood, a frequency or repetition of a keyword or mood throughout several entries, percentage of a color used in a drawing, and so forth. Alerts may be provided within the interfaces of the user-side applications of system 100 and may also be provided or transmitted as push notifications on a supervisory user's mobile or personal device. The alert functionality may further be enhanced by analytics module 106.

Analytics module 106 may be adapted to analyze subject users' entries and provide comprehensive analysis and insights to supervisory users of the subject users' moods and mental health over time. For example, analytics module 106 may collect data regarding the date, length, frequency, and relative amount of usage of entry module 102, the usage and selected exercises of exercise module 104, and so forth. Analytics module 106 may further utilize speech-to-text functionality so as to transcribe the contents of the audio and video recordings of journal entries made by the subject user or by a supervisory user interacting with a subject user.

Analytics module 106 may further utilize artificial intelligence algorithms to analyze the transcribed text of entries and determine the existence of any desired keywords or alert words in the entries. For example, alert words may include such terms as “sad”, “angry”, “mad”, “upset”, “cry”, “bully”, “nightmare”, and so forth. Alert words may also include terms such as “happy”, “joy”, “fun”, “friend”, and so forth. The AI may populate a set of “alert word suggestions” or concern marker suggestions. The AI suggestions may be determined by, for example, the AI analyzing a plurality of subject users and/or anonymized user analytics from the plurality of subject user's and using that information to determine common keywords that are associated with predictive analytics, trendlines, or particular patterns. A pre-defined set of alert words or sequences of alert words may be provided, and a supervisory user may add and remove alert words as desired to customize the alert functionality for a particular subject user. For example, a supervisory user may recognize that a subject user has a reaction to a certain person's name or a certain topic. Such alert words may then be added to the set of alert words or set of sequences of alert words.

Analytics module 106 may be adapted to notify a supervisory user based on an occurrence of markers. Markers may include concern markers and positive markers. For example, a concern marker may be a “negative” alert word or a lack of a “positive” alert word, while a positive marker may be a “positive” alert word or a lack of a “negative” alert word. Words may be automatically assigned a particular connotation by AI analysis of the plurality of users, or may be set by the user or supervisory user. As a further example, if a certain alert word or sequence of alert words occurs more than an indicated number of times within a particular timeframe, or with a higher than indicated frequency, analytics module 106 may alert the user. Conversely, if a certain alert word or sequence of alert words occurs less than an indicated number of times within a particular timeframe, or with a lower than indicated frequency, analytics module 106 may likewise alert the user. For example, a supervisory user may be alerted if a subject user used the word “mad” three times consecutively, or used the word “happy” less than twice a week. Furthermore, analytics module 106 may be adapted to notify a supervisory user based on an occurrence of concern markers such as a particular color in a subject user's drawing. For example, if a certain color is used in a large percentage of a drawing, and/or if such usage occurs more than an indicated number of times within a particular timeframe, or with a higher than indicated frequency, analytics module 106 may alert the supervisory user. For example, a supervisory user may be alerted if a subject user used the color red for 50% or more of a drawing in four or more entries. In some embodiments, the supervisory user may be presented AI suggested alert word connotations (positive, negative, or neutral) to either adopt or adjust. For example, a child may really like the idea of monsters under the bed and talk about that often even if most children their age may be scared of monsters under the bed. This process also helps to further train AI suggestions improving specificity and lessoning margin for implicit bias.

Analytics module 106 may further track and correlate other aspects of a subject user's interaction with system 100. The subject user's moods logged in association with entries may be analyzed for frequency, repetition, and correlation with other aspects of the subject user's entries. Notes and attachments associated with entries may further be analyzed so as to determine correlations between moods, input content, and external influences on the subject user.

Analytics module 106 may utilize several methods and algorithms, for example AI or machine-learning algorithms, to perform the analysis of entries. The machine-learning system may make decisions based on a plurality of data, such as entries, compiled from a plurality of users. The machine learning system may update a baseline profile over time based on new analysis of the plurality of users. The machine learning system may determine thresholds for various metrics and may update those thresholds over time corresponding with changes in the data obtained from the plurality of users. The thresholds may be utilized for a variety of purposes in system 100. For example, certain mood indicators, inputs, “flags”, or behavioral information associated with a user may be understood by the system 100 to exceed a threshold, such a negativity threshold or self-care threshold. In such circumstances, the exceeding of the threshold may be used as a trigger to have the system automatically push notifications (for example to a supervisor or caretaker) or can be utilized by the system to provide or transmit alternative prompts, interfaces, or characters to the user. In an example where a negativity threshold is exceeded, a user may be provided with an animated character to guide or instruct the user on some positive or positivity exercises or in the case of an adult presenting with extreme anxiety, de-escalation stress navigation funnels may be utilized. Such automatic or dynamic action may also coincide with transmitted notifications to a supervisor or caretaker in order to ensure that appropriate attention is provided to the user or appropriate care is provided directly to the user. Additionally, the supervisory user may have the ability to adopt or adjust AI suggested thresholds as they dynamically populate, as well as manually adjust or create, thresholds for customization. Further, in some embodiments, when a threshold is exceeded, the system 100 may also initiate hardware action, for example automatically activating audio and/or video recording in order to obtain more user information and/or provide further information to a supervisor or caretaker. Such actions could also include dynamically connecting a user to a supervisor or caretaker for real time, synchronous communication or interaction between the two. In further embodiments, in such examples where an emergency threshold is reached or immediate intervention alert words are triggered, such as involving active self-harm, emergency push notifications may be sent/transmitted, or preauthorized permissions may be accessed, dynamically connecting users or supervisory users to emergency services.

These methods and algorithms may utilize Neural Networks and Natural Language Processing, such as but not limited to, Artificial Neural Networks, Convolution Neural Networks, Recurrent Neural Networks, Lexical or Morphological Analysis, Syntax Analysis, Semantic Analysis, Discourse Integration, Pragmatic Analysis, and other Deep Learning Models as well. In some embodiments, the machine-learning system, in addition to full plurality, may also utilize furcated datasets or structured data-set isolations with transparently stated variables specific to the use case to help remove bias from the interpretative structures. For demonstrative purposes, the entire system may be viewed as a data ecosystem with encompassed data biomes that are interconnected but not always relevant to specific output determinations. For example, in the use case of determining behavioral assessment outputs of children or adults, the plurality of data of children may not be relevant to assess in the plurality data of adults for specific behavioral outputs, but may be relevant in the assessment of regressive or digressive behaviors or trendlines and patterns of recursive behavioral outputs over time. Additionally, in some embodiments, a supervisory user or trained AI may determine the dataset furcations or isolations as well as AI may present dataset furcations to be adopted by the supervisory user. Moreover, trained AI correlations may be presented and displayed utilizing the dataset furcations which also may be adopted by the supervisory user. In some embodiments, supervisory user adoption of AI correlations, categorizations, etc. may lesson systemic bias, improve customization and outputs, as well as improves AI training. Furthermore, in some embodiments the machine learning system may utilize and interpret decisions including the determinations or deterministic scenarios of semantic reasoners and likelihood ratios, such as but not limited to, plurality trendline match ratios, baseline conflict probability ratios, sequences of alert word progression reasoners, potential heuristic or metaheuristic approaches or other more robust algorithms curated to the plurality of data. For example, analytics module 106 may be adapted to detect colors, shapes, and subject matter of drawn entries, as well as alert words, common patterns of words, sequences of words, repetition of particular words or phrases, or matches to other trendlines within the subject user or among the plurality of users. In some exemplary embodiments the analytics module 106 may be able to identify connections between shapes, colors, and subject matter of drawn entries with specific subject matter. In some exemplary embodiments, analytics module 106 may further be adapted to determine tonal connotation and/or behavioral interpretation of an entry by detecting facial expressions, body language, and voice intonations in video and/or audio recorded entries, so as to provide further insight on the emotions of the subject user. Tonal or behavioral connotation may be facilitated by utilizing historical data from all or subsets (such as cohorts) of users of system 100. This can be further analyzed and processed to provide connotation by evaluating the historical data in comparison with long or short term medical treatment and known outcomes. For example, the analytics module 106 may learn that some words or inputs in the historical data and may compare them with later known effective treatment plans, known diagnoses, or other known medical or psychological outcomes to learn and effectively determine tonal or behavioral connotations. In some exemplary embodiments, the analytics module may utilize motion tracking and capture, such as but not limited to human motion recognition, human gesture recognition, and facial emotion recognition. In some exemplary embodiments, analytics module 106 may further be adapted to detect a subject user's cognitive dissonance or distortions throughout an entry. Examples of cognitive dissonance include, but are not limited to, all or nothing thinking, over-generalizing, jumping to conclusion, personalization, absolutism, etc. In further embodiments the analytics module 106 may detect other cognitive biases, fallacies, illusions, or effects. Examples of cognitive biases, fallacies, illusions or effects include, but are not limited to, confirmation bias, spotlight bias, negativity or positivity bias, ad hominem fallacy, red herring fallacy, bandwagoning effect, anchoring effect, framing effect, ostrich effect, clustering illusions, frequency illusions, and so forth. In yet further exemplary embodiments, analytics module 106 may be adapted to utilize artificial intelligence for predictive analytics. Analytics module 106 may further analyze anonymized data from a plurality of user accounts of system 100 so as to predict patterns of concern or positive mental health trajectories. Furthermore, system 100 may utilize artificial intelligence to detect early-stage issues, protect subject users in dangerous situations or settings, and to predict common data trends with varying early-stage mental health diagnoses. Over time, such functionality may be adapted to analyze entries to detect early stages of abuse, data commonalities preceding a mental health diagnosis, and other predictive patterns related to mental health and wellness.

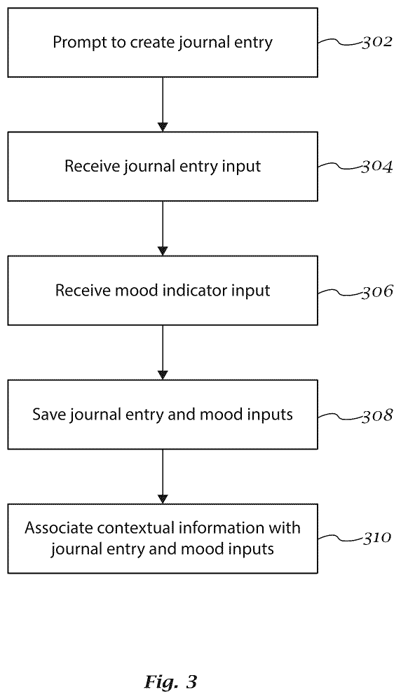

FIG. 3 shows an exemplary method 300 for receiving mental wellness information. At step 302, a prompt to create an entry may be presented to a user, such as a subject user or a clinical professional. At step 304, input information for the entry may be received, including drawing input, text input, video input, and/or audio input. Input information may include journal entry information and/or session entry information. At step 306, a mood indicator may be received and associated with the input information for the entry. The mood indicator may include a description of the mood and/or a color associated with the mood. At step 308, the entry and associated mood indicators may be saved to data storage. In some embodiments, AI algorithms may further be used to analyze the entirety of the entry to determine mood. Optionally, at step 310, contextual information may be received from a supervisory user and associated with the entry in data storage. The contextual information may include notes, attachments, forms, and/or flags. Flags may include, for example, specifying when cognitive dissonance or distortions, bias, fallacies, illusions, or effects are found. In some embodiments flags the user-behavioral baseline may be utilized to detect agreements or conflicts in user-created inputs. At step 312, the recorded inputs, such as the drawing, audio, and video inputs, may be transcribed and associated with the entry in data storage.

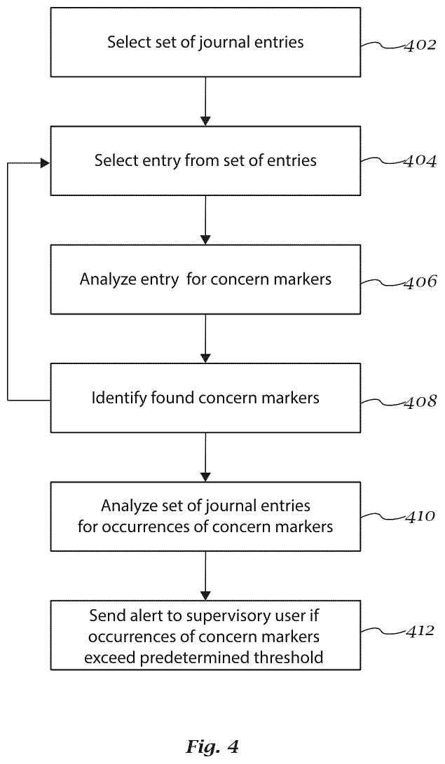

FIG. 4 shows an exemplary method 400 for analyzing mental wellness information. At step 402, a set of entries may be selected in data storage. At step 404, an entry from the set of entries may be selected. At step 406, the selected entry may be analyzed, for example by an AI algorithm, for markers, including concern markers and positive markers. Such markers may include, for example, the presence of alert words and/or sequences of alert words in the transcription of a recorded input, the presence of certain colors in a drawing input, a certain mood indicator, a color associated with a certain mood, and so forth. When found, the markers may be identified within the entry, at step 408. For example, alert words may be highlighted in the transcription of the recorded input. Steps 404-408 may be repeated for each entry in the set of entries. Such mood indicators or alert words or sequences of alert words may further be used in forming a threshold, as noted above.

At step 410, the plurality of entries may be analyzed for occurrences of markers or sequences of markers within the plurality of entries. The analysis may be based on several factors, such as frequency of occurrence of markers or sequences of markers within a predetermined time frame, absolute number of occurrences of markers or sequences of markers, a percentage of a marker or sequence of markers within an input, as well as correlations between occurrences of markers or sequences of markers and occurrences of other terms in the entries and correlations between occurrences of markers or sequences of markers and content of notes and attachments. It is understood that the correlations may be automatically or dynamically performed as the entries are made. Further, correlations may be performed based on historical data associated with the user or based on a cohort of users in the system 100. For example, if the user is a male, aged 13, historical data of other users with similar ages and demographics, users in a similar/same geographic area, users in a same school or school district, users in a same school grade, or the like, (e.g., users in a cohort) may be automatically processed and interpreted with respect to an individual user to determine correlations. If the occurrences of markers exceed a predetermined threshold, an alert or notification may be sent or transmitted to a supervisory user, at step 412. Further, in some embodiments, if users in a certain cohort, for example users in a same school or same school district, are determined to have different or other outlying correlations from other such cohorts, then further, broader scale, alerts or notifications can be provided, for example to appropriate school or district personnel. Additionally, correlations may be adjusted based on any of a variety of factors associated with a cohort, for example a cohort with urban students versus one with rural students.

In some exemplary embodiments user-created inputs to various prompts may be utilized to establish a user-behavioral baseline. The prompts may be structured to receive submissions on identified detectible moods, and inputs may be at least one of, but not limited to, video entries, text entries, drawn entries, or mood entries. The user-behavioral baseline may be used to compare a user's future inputs against their own generated baseline, or against AI generated general baselines created from, for example, a plurality of similar subject users, or generalized subject user behaviors.

A testimony analysis report may be used to help analyze a subject user's transcriptions and other sources to aid in baseline collection. The testimony analysis report may include a plurality of primary and/or secondary sources. Primary sources may include, but are not limited to, original testimony, compiled video analysis report, compiled transcription analysis report, transcription vs. video analysis report, and/or physical sensor report of testimony capture. Secondary sources may include, but are not limited to, compiled evidence reports and contextual analysis, compiled witness reports and contextual analysis, and/or compiled expert reports and contextual analysis.

In some exemplary embodiments structured prompts may be used for assessment and funneling weight. Structured prompts may include, but are not limited to, stress navigator prompt structures, additional “worksheet structures”, CBT, DBT, and ACT techniques, emotion identification or regulation, defusion techniques, cognitive restructuring techniques and/or prompts or worksheets specific to certain mental illnesses such as anxiety, addiction, or PTSD. The structured prompts and inputs may be analyzed by the user and/or AI and may be further analyzed along with other notes and attachments to determine correlations between moods, input content, and external influences on the subject user.

In some exemplary embodiments, due to the inconsistent and non-linear nature of human thought, the subject user may assess and contribute to the weighting of their own data. The AI may take into account the subject user's weighting in order to focus its analysis, lessen the margin of missing key points, and/or assess or progress system accuracy. In some embodiments metrics of the subject user's thoughts as compared to the AI input patterns may indicate deeper or other issues. This dynamic weight adjustment by the AI can be performed automatically, and can be based on both real time or longer term analysis of historical user data and inputs, based on a comparison of user data with cohort weighting data, or based entirely off of an analysis and comparison with cohort data, for example in situations where there may not be sufficient user historical data to provide appropriate AI learning. Additionally, in further embodiments of advanced clinical use, the subject user and supervisory user would also have ability to provide assessments to the feedback loop. The subject user assessment of input weight hierarchy would allow the subject user to confirm or deny accuracy of data such as standing beliefs and the importance or weight of standing beliefs. The user weighted data may also be separate from the data which is assessed by the AI and systems for other analysis such as frequency of occurrence. These separate data sets may be processed for deeper insight to either be adopted by the ecosystem of larger system data sets (improving accuracy, allowing the system to focus on higher weighted items, lessening the margin of error or missing key points, and providing a point to assess program system accuracy). Or conversely the subject user assessments will also provide information and metrics of how the subject user perceives their own thoughts. This will add complexity to the data set and the AI interpretation of input patterns which may indicate deeper issues not directly recognized by the subject user. This is especially important in examples of complex interaction such as psychosis, schizophrenia, etc. In some embodiments weight given by supervisory input data may be separated or noted by the system, particular where the input data flags conflicts. These subject user and supervisory user weight assessments will greatly add in iterative machine learning optimization algorithms and helping the AI models learn over time in a way that accounts for direct human feedback providing new and greater levels of complexity and insight which without such a system would not be possible to comprehend.

In some exemplary embodiments there may be a contextual mesh, wherein contextually relevant past information may be inserted into a timeline in order to provide additional information on items within the timeline. The contextual mesh may contain context containers, which may be, for example, a transcription, drawing, detention slip, earlier video, health form, psychiatric report, or assessment quiz, and may place these context containers where relevant in a live timeline search. In an exemplary embodiment, a live timeline search may bring up a particular phobia (e.g. spiders), the contextual mesh may then place past videos or drawings related to the phobia in the timeline to provide larger context. In some exemplary embodiments the contextual mesh may further contain contextual alerts, correlations, and/or peripheral data sets.

In some exemplary embodiments user's may be able to refine suggested contextual mesh clusters in order to help find relevant contextual connections. For example, a child may call spiders “crawlies”, so the user may manually input that connection in order to help refine the contextual mesh searches for that subject user. These refinements may be input by a supervisory user or adopted by AI suggestion.

In some exemplary embodiments a color to AI mood “heat map” may be created to visualize to the user their mood over a period of time. The AI may assign specific percental of color to specific moods detected by the AI, then may plot the color on a corresponding plotline, the heat map may also convey other information for example the intensity of the mood. In other embodiments the AI may create a keyword heat map by selecting a particular keyword, assigning colors to the emotions and/or sequences of markers, that were present when discussing the key word to create a relationship with the keyword. In further embodiments the heat maps overtime may provide visual insight of the overarching complex analytics and provide a method to visually explore the dataset and thus relationships to specific time periods, keywords, etc. In the specific case of mood heatmapping, this analysis may also be collected independently by both AI mood indicators and supervisory users assessments. The subject user's heat map from submitted mood assessment may then be cross correlated with the AI assessment and supervisory user's assessment of the subject user's mood. This cross assessment would provide further insight into a subject user's perceived internal moods, versus what is presenting externally.

Logged usage data may further be used to provide trends and patterns regarding a subject user's interaction with system 100. The usage of the entry and exercise modules of system 100 may be logged, for every instance of use of the application. The logged data may then be displayed, for example as a graph that shows the amount of usage of the entry module and each exercise of the exercise module over time. The moods entered by the subject user at every instance of use of system 100, and/or those detected by AI algorithm analysis, may be logged and displayed as a graph showing the occurrence of each mood over time. Supervisory users may utilize such graphs to find trends and patterns that correlate with external stressors and points of concern, and to reinforce areas that improve the mental wellness of the subject user. Examples of logs or reports include, but are not limited to, session logs, mood and usage graphs, session reports, overall mood results, belief results, trigger logs and analytics, and/or shown vs. shadow self assessments. User's may be able to navigate through and visualize connections between display logs, for example by using navigational features such as pinch and zoom on a visualized display of the logs and analytical reports. Additionally, alert words or sequences of alert words with frequency of occurrence may be represented by scale in web maps and connected associated keywords or sequences of keywords. These web maps may have color view options such as neutral color (to focus on frequency and correlation), connotation coloring, mood heat map coloring, and color blind options.

Furthermore, logged repetition or sequences of prompt inputs may be used to track and analyze over time. For example, there may be a “brain-backup” system which may act as a backup of memories to show similarity or degradation of recollection over time, for example in the treatment or study of a user with Alzheimer's or other degenerative brain conditions, or when comparing testimony over time to see whether a user's version of events conflicts or is supported by past recollections. Other examples including but not limited to, assessing resilience/coping skills of children over time, tracking and supporting educational and extracurricular interests, assessment of rumination cycles, depression or anxiety management over time, nurturing the development of positive mental health practices/trajectories over time and so forth.

In an exemplary use case, a school district may be able to see an admin map displaying alert words for the user base of the school. One visualization may show negative example alert words and may be used to identify, for example, issues with homework load, school lunches, playground safety, etc. Another visualization may show positive example alert words and may be used to identify well liked programs, teachers, or learning plans/subjects.

In some embodiments the system may output other automations regarding predictive analytics, trendlines, and patterns with its supporting data to look at specific illnesses or other predictive analytics such as highlighting areas of concern. Furthermore, supervisory users may be alerted about specific trendlines and patterns, especially if certain thresholds are crossed and need immediate intervention such as in the case where suicidal ideation is detected. For example, a supervisory user may be alerted of a subject user exceeding a threshold and an automation output offering the supervisory user's pre-set or customized treatment suggestions may be sent to the subject user. These automations or outputs may range from supervisory user treatment comments to beneficial reading materials or exercises, to direct access to necessary doctors and immediate intervention if applicable.

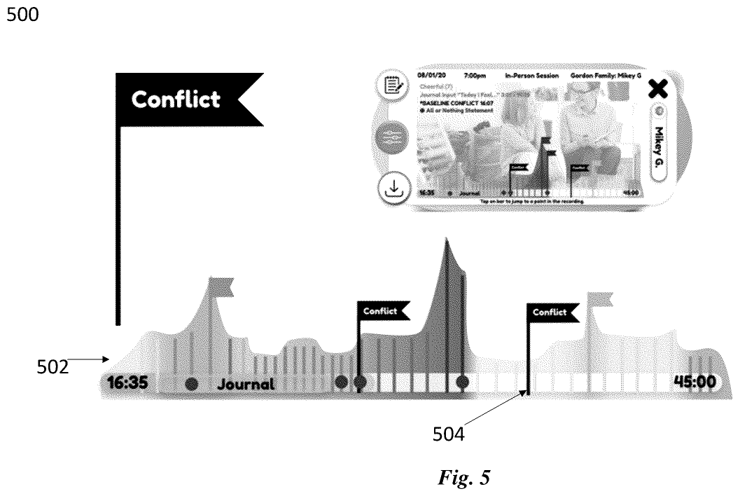

FIG. 5 may show an exemplary user timeline with conflicts tagged 500. The exemplary user timeline with conflicts tagged 500 may include a user timeline 502, which may show entries over a period of time. The user timeline 502 may further show user emotions and intensity over time through, for example, color or height of the graph. The user timeline 502 may indicate where conflicts 504, such as cognitive dissonance or distortion, biases, fallacies, illusions, or effects are detected. The conflicts 504 may be indicated by, for example, markers such as flags, or other visual or auditory indicators.

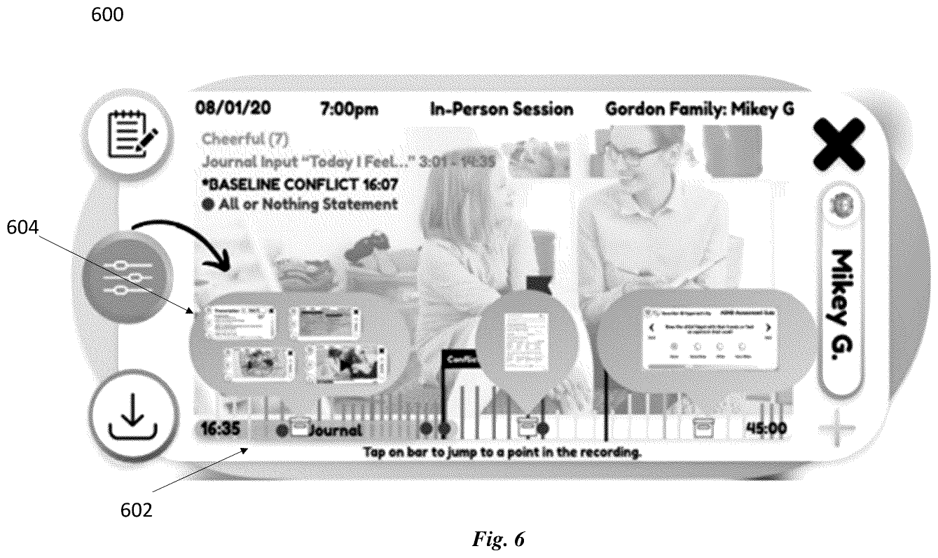

FIG. 6 may show an exemplary contextual mesh timeline 600. The contextual mesh timeline 600 may include a user timeline 602, which may show entries over a period of time. The user timeline may indicate where contextual information 604 has been found, such as through a visual display or icon on the user timeline 602. The contextual information 604 may further be displayed or linked to from the user timeline 602.

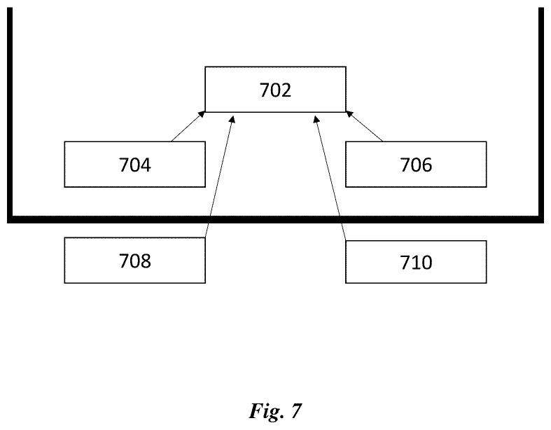

FIG. 7. may show an exemplary behavioral baseline structured prompt map 700. The behavioral baseline structured prompt map 700 may include a subject user behavioral baseline 702. The subject user behavioral baseline 702 may be created from a plurality of subject user prompt structure baseline collections 704 and a plurality of AI generated mood marker subject user collections 706. The user behavioral baseline 702 may further take in plurality of other similar user profiles 708 and/or take in a plurality of all other user profiles 710.

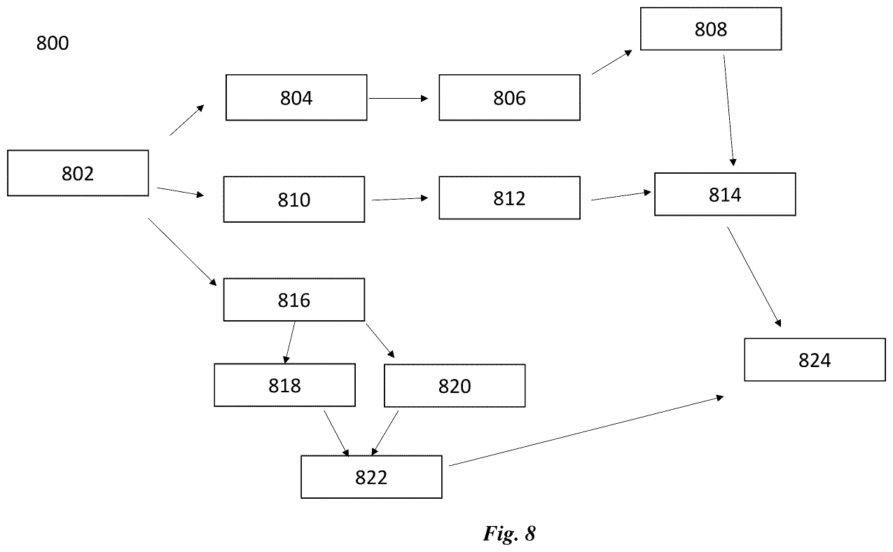

FIG. 8 may show an exemplary user-behavioral analysis system 800. The analysis system 800 may include original testimony 802. Analysis may be done on the original testimony by, for example, a plurality of APIs 804, which may control a plurality of sensors 806. The plurality of sensors 806 may include, but are not limited to, heart rate sensors, brain activity sensors, eye focus sensors, or other physical sensors. The data from the plurality of sensors 806 may be used to generate a physical sensor report of testimony capture 808. Further analysis may be done on the original testimony 802, for example video analysis 810. The video analysis 810 may include mood/response analysis 812, which may be to, for example, a self-baseline, a comparable average baseline, or to a general human baseline. The mood/response analysis 812 may be combined with the physical sensor report 808 to create a video analysis report 814. Further analysis may be done on the original testimony 802, for example, transcription analysis 816. The transcription analysis may include AI tone analysis 818 and/or cognitive dissonance analysis 820, which may be to, for example, a self-baseline, a comparable average baseline, or to a general human baseline. The AI tone analysis 818 and/or cognitive dissonance analysis 820 may be used to create a transcription analysis report 822. The transcription analysis report 822 may be combined with the video analysis report to create a transcription vs. video analysis report 824.

The embodiments disclosed herein can therefore provide a means of expression for a subject user, where the subject user may be comfortable in expressing themselves in ways that they may not feel comfortable expressing to a supervisory user and to learn healthy exercises and mindfulness techniques. The embodiments disclosed herein can further provide a means for parents, caretakers, and professionals to obtain insight into the day-to-day feelings of the subject user, to understand correlations between the subject user's moods and external stressors, and to obtain context for the subject user's moods and emotions, and obtain insight without bias and prejudice of analysis.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A method for data transmission, comprising:
inputting an entry from a user through an interface on a user device;
transmitting the entry to a remote data storage;
storing the entry with user historical data;
analyzing, with a processor, the entry;
performing correlations on the entry using at least the user historical data;
determining at least one of entry classifications, baselines, or a new input prompt as a result of correlated entry data;
transmitting the at least one of entry classifications, baselines, or new input prompt to the user device; and
displaying the at least one of entry classifications, baselines, or new input prompt on the user device.

2. The method of claim 1, further comprising performing correlations on the entry using cohort data.

3. The method of claim 1, further comprising transmitting a notification to a supervisory user as a result of the correlated data.

4. The method of claim 1, further comprising determining and generating one or more threshold values associated with at least one of historical user data and cohort data.

5. The method of claim 1, further comprising transmitting a notification to a supervisory user as a result of the correlated data, wherein the notification contains data for threshold values to be adopted or adjusted by the supervisory user.

6. The method of claim 5, wherein the input prompt is dynamically generated in response to the one or more threshold values being exceeded.

7. The method of claim 1, further comprising weighting a value associated with the user entry with respect to a plurality of entries in the user historical data.

8. The method of claim 1, further comprising weighting a value associated with the user entry with respect to a plurality of entries of cohort data.

9. The method of claim 6, further comprising transmitting a displayed notification that the threshold value is exceeded.

10. The method of claim 6, further comprising automatically activating hardware on the user device when one or more threshold values are exceeded.

11. The method of claim 10, wherein the hardware comprises at least one of a microphone and camera.

12. The method of claim 6, further comprising initiating a synchronous communication session between the user device and a supervisory user or dynamically populating a response between the user device and the supervisory communication device when the data exceeds the threshold.

13. A communication system, comprising:
a user input device configured for wireless communication to at least a remotely located data storage;
a supervisory communication device configured for wireless communication to at least the remotely located data storage; and
a processor configured to:
receive input data from the user device;
analyze input data;
compare the input data with one or more of historical user data and cohort data;
determine when the input data are at least one of entry classifications, baselines, or exceeds a threshold;
generate new entry classifications, baselines, or an input prompt when the data exceeds the threshold;
transmit at least one of entry classifications, baselines, or the input prompt to the user device;
classify the at least one of entry classifications, baselines, or input data; and
initiate a synchronous communication session between the user device and a supervisory user or generate a dynamically populated response that is transmitted to the user device from a supervisory user when the data exceeds the threshold.

14. The system of claim 13, further comprising at least one of a camera and a microphone on the user device.

15. The system of claim 14, further comprising at least one of video and audio data recorded by the at least camera and microphone, wherein the recorded data is automatically transmitted to a remotely located storage device.

16. The system of claim 13, further comprising trained artificial intelligence that determines when the input exceeds the threshold.

17. The system of claim 16, wherein the trained artificial intelligence is trained using at least one of the historical user data and cohort data.

18. The system of claim 16, wherein the trained artificial intelligence generates outputs and trend lines using at least one of the historical user data and cohort data.

19. The system of claim 16, wherein the trained artificial intelligence outputs and trend lines using at least one of the historical user data and cohort data may be displayed as reports.

20. The system of claim 13, further comprising generation and display of an animated character or avatar that communicates the input prompt or information input on the user device via the supervisory user.
